(19) United States
(12) Patent Application Publication
(10) Pub. No.: US 2016/0015157 A1
(43) Pub. Date: Jan. 21, 2016

(54) COMPOSITIONS AND METHODS FOR ARRANGEMENT OF SURVIVAL MATERIALS

(71) Applicant: Karim J. Nouri, Reseda, CA (US)

(72) Inventor: Karim J. Nouri, Reseda, CA (US)

(21) Appl. No.: 14/805,253

(22) Filed: Jul. 21, 2015

Related U.S. Application Data
(63) Continuation-in-part of application No. 14/078,180, filed on Nov. 12, 2013.

(51) Int. Cl. A45F 3/16; A61F 17/00; A45F 3/04

(52) U.S. Cl. CPC A45F 3/16; A45F 3/04; A61F 17/00; A45F 2003/166

(57) ABSTRACT

A survival kit to include a specially designed and arranged backpack with emergency contents is disclosed. The backpack is configured with a plurality of compartments wherein a hydration bladder having a tube for accessing water therein is provided in the first compartment. Importantly, the kit has a pair of goggles and a plurality of breathing masks particularly useful for urban disasters such as an earthquake or a terrorist attack. Similarly, work gloves are included to assist in rescue and recovery operations. Additional items configured to the kit are a lightweight, fire resistant, tube tent being devoid of tent poles and emergency drinking water germicidal tablets, just to name a few.

### PRIORITY CLAIM

This patent application is a Continuation-In-Part application containing subject matter claiming benefit of the priority date of U.S. patent application Ser. No. 14/078,180 filed Nov. 12, 2013 which claims the benefit of the priority date of U.S. Provisional Patent Application No. 61/725,264 filed on Nov. 12, 2012.

### BACKGROUND OF THE INVENTION

1. Field of the Invention

The present invention generally pertains to survival items and methods of ensuring prolonged survival following a disaster event. More particularly in a preferred embodiment, the invention relates to a backpack optimally configured in order of time criticality and fitted with food and water rations in addition to first aid, shelter, tools and other survival items.

2. Description of the Prior Art

Recent natural disaster events, such as hurricanes Katrina and Sandy, have brought a renewed interest in emergency preparedness and maintaining survival without electricity or water (or possibly shelter). Other possible disaster scenarios have also been in the human conscience such as a terrorist attack, nuclear holocaust or a cataclysmic cosmic event. Even before Katrina and Sandy, various wearable or portable apparatuses/kits had been proposed for emergency preparedness. One example was invented by Perry, entitled “Emergency Preparedness Vest Apparatus,” U.S. Pat. No. 5,361,412 and was awarded patent protection in 1994.

According to Perry, recent events, such as earthquakes, fires, etc., have demonstrated the practicability and the advisability of being prepared for various types of emergencies. It has been suggested that a seventy-two hour time period, or three days, is sufficient for the re-establishment of normal facilities after a disaster of one kind or another. That is, people should ideally be able to maintain themselves for a period of three days in the event of an emergency situation.

Another more recent survival pack example was proposed by Marcon, entitled “Personal Survival Vestpac,” U.S. Pat. No. 6,484,912. This particular design provides protection from exposure and further includes specialty items that seem suited for survival for the outdoor adventure enthusiast. As an example, a radio beacon and insect repellant are configured to the wearable apparatus. The present invention however, is more specifically suited to the average urban or town citizen following a disaster event.

An additional example was proposed by Jones, entitled “Experienced Backpacker,” U.S. Pat. No. 6,314,579. This example provides a backpack the purports versatility as it can be used as an emergency stretcher and is even fitted with stretcher poles. The design further includes items that are mainly first aid in nature configured to the backpack. The solution appears to be designed for outdoor emergency responders such as ski patrol, park rangers, and the like.

While the previous emergency type packs and kits seem suitable for their intended purposes, a need remains for a solution geared more toward the average citizen caught in an emergency and less geared toward the outdoor enthusiast caught in an avalanche or other event in remote country. In light of the aforementioned, it is an object of the present invention to design a backpack having compositions of emergency items configured thereto, having useful and handy items, particularly useful in the wake of a natural disaster.

It is still further an object of the present invention to provide an emergency survival backpack that carries provisions for approximately three days and providing extra room for special inventory while not exceeding a maximum comfortable weight. It is further an additional object of the present invention to provide a specific pre-configured optimum load out to average citizens without experience and expertise in emergency situations. It is yet further still further an object of the present invention to provide emergency preparedness training to the general public.

It is yet another object of the present invention to provide an active shooter response system including tools and gear necessary for law enforcement to approach an active shooter scenario. It is an additional object of the present invention to provide an active shooter response system wherein the response system provides first responders with the equipment to engage an assailant, manage a scene, facilitate civilian evacuation and render emergency first aid care on site efficiently due to the “first order retrievability” of the system’

### BRIEF SUMMARY OF THE INVENTION

The present invention specifically addresses and alleviates the above mentioned deficiencies associated with the prior art. More particularly, the present invention in a first aspect, is an a survival kit comprising a specially designed and arranged backpack, the backpack comprising: a plurality of compartments with a first compartment being closest to a wearer's back, the first compartment including a hydration bladder having a tube for accessing water therein without having to remove the bladder from the first compartment; a second compartment including a plurality of water packets and food rations; and a third compartment including a pair of goggles and a plurality of breathing masks.

The invention in this aspect also includes a fourth compartment adjacent to the third compartment, the fourth compartment containing only a first aid kit wherein the fourth compartment is ideally sized for a snug fit for the first aid kit and located to assist in quick and easy access. A fifth compartment is located underneath the fourth compartment, wherein the fifth compartment includes work gloves to assist in rescue and recovery operations and is likewise in a convenient accessible location.

Still further, the invention in this aspect is characterized wherein the fifth compartment includes a compact, hooded poncho; and loops configured to an outer portion of the fifth compartment, the loops retaining a flashlight. The invention also including may other survival items such as: a lightweight, fire resistant tube tent, the tube tent being devoid of tent poles; a plurality of trash bags tightly rolled to conserve space, a nylon cord, a plurality of emergency drinking water germicidal tablets, a plurality of glow sticks, a small saw, a small roll of duct tape, a tinder kit with lighting instructions, a plurality of waterproof matches, a pair of ear plug, a pen and a paper, a reflective and insulative tarp, a compass, a plurality of hand warmers, a survival guide book, a mirror, a plurality of batteries; and an insulated pocket sized sleeping bag offering protecting from rain, wind and cold temperatures.

Further yet, the invention in another aspect is characterized as having equipment to engage an assailant, manage a scene, facilitate civilian evacuation and render emergency first aid care on site, including but not limited to, ammunition magazine readily available, restraints, masks, lights and various multi tools, a pole-less litter, compact survival kit and compact trauma kit.

The invention can be better visualized by turning now to the following drawings wherein like elements are referenced by like numerals.

These, as well as other advantages of the present invention will be more apparent from the following description and drawings. It is understood that changes in the specific structure shown and described may be made within the scope of the claims, without departing from the spirit of the invention.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a side aspect perspective view of a first preferred embodiment of the present invention;

FIG. 2 is a is top view of the first preferred embodiment;

FIG. 3 is a top view of a cardboard box containing water and food rations configured to the first preferred embodiment;

FIG. 4A is a first perspective view of a smaller provisions pack for one of the compartments of the present invention;

FIG. 4B is a second perspective view thereof in an open position;

FIG. 5A is a front plan view of the first preferred embodiment:

FIG. 5B is a first perspective view from a front aspect illustrating contents of a fifth compartment of the present invention; and

FIG. 5C is a second perspective view from a front aspect illustrating contents of a third compartment of the present invention.

FIG. 6 is aspect perspective view of a second preferred embodiment of the present invention.

FIG. 7 is an alternate perspective view of the second preferred embodiment.

FIG. 8 is a close-up view of weapons magazine pouches of the present invention.

FIG. 9 is a close-up view of the flexible restraints with holster.

FIG. 10 is a close-up view of the external compartments for a folding knife with belt cutter and glass breaker of the present invention.

FIG. 11 is a top view of a first compartment for door stops and stays in an open position.

FIG. 12 is a close-up view of a second compartment for additional supplies in an open position in the second preferred embodiment.

FIG. 13 is a close-up view of a third compartment for further additional supplies including a compact survival kit and trauma kit in an opening position in the second preferred embodiment.

FIG. 14 is a close-up view of the third compartment shown in FIG. 8 in an open position with a detailed view of the rip-away EMT pouch in the second preferred embodiment.

FIG. 15A is a perspective view of the rip-away EMT pouch removed its compartment.

FIG. 15B is a top view of a first side of the rip-away EMT pouch in an open position.

FIG. 15C is a close-up view of the first side of the rip-away EMT pouch in an open position shown in FIG. 15A.

FIG. 15D is a close-up view of a second side of the rip-away EMT pouch in an open position shown in FIG. 15A.

FIG. 16A is a close-up view of a fourth compartment for holding still further supplies including a portable litter, headlamp and paracord.

FIG. 16B is a close-up view of the portable litter stored in the fourth compartment in the second preferred embodiment.

### DESCRIPTION OF PREFERRED EMBODIMENTS

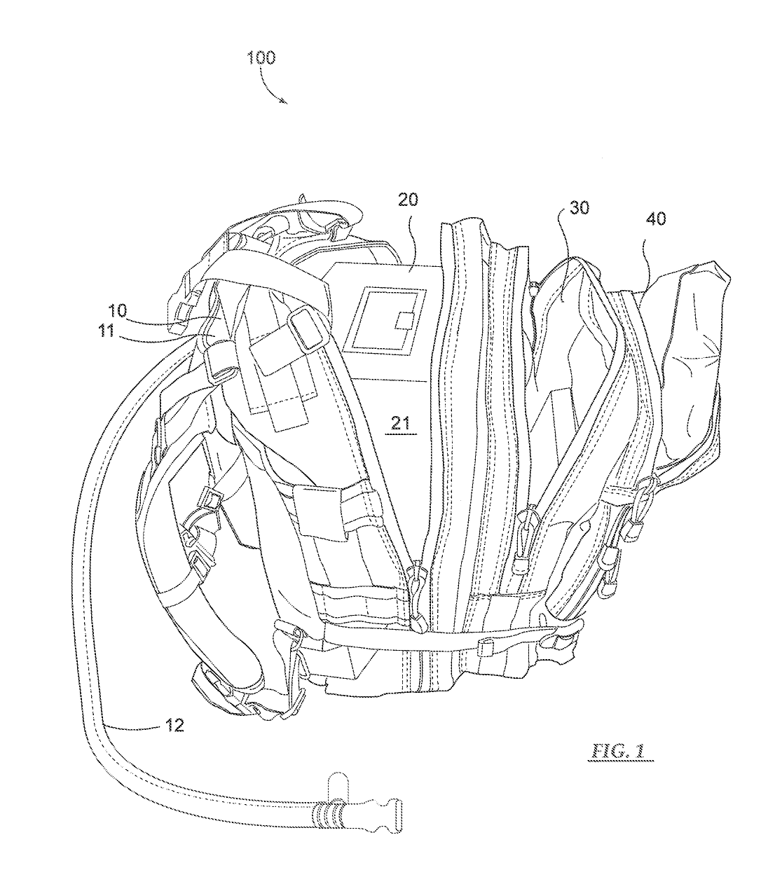

Initially with regard to FIG. 1, a side aspect view of an emergency backpack 10) is provided showing four adjacent (first 10 second 20 third 30 and fourth 40) compartments with a first 10 being closest to a wearer's back and other compartments 20, 30, 40, adjacent thereto. The first compartment 10 holds a hydration bladder 11 having a tube 12 for accessing water therein without having to remove the bladder 11 from the compartment 10. In a preferred embodiment 10, the bladder 11 holds approximately 2.5 liters of liquid.

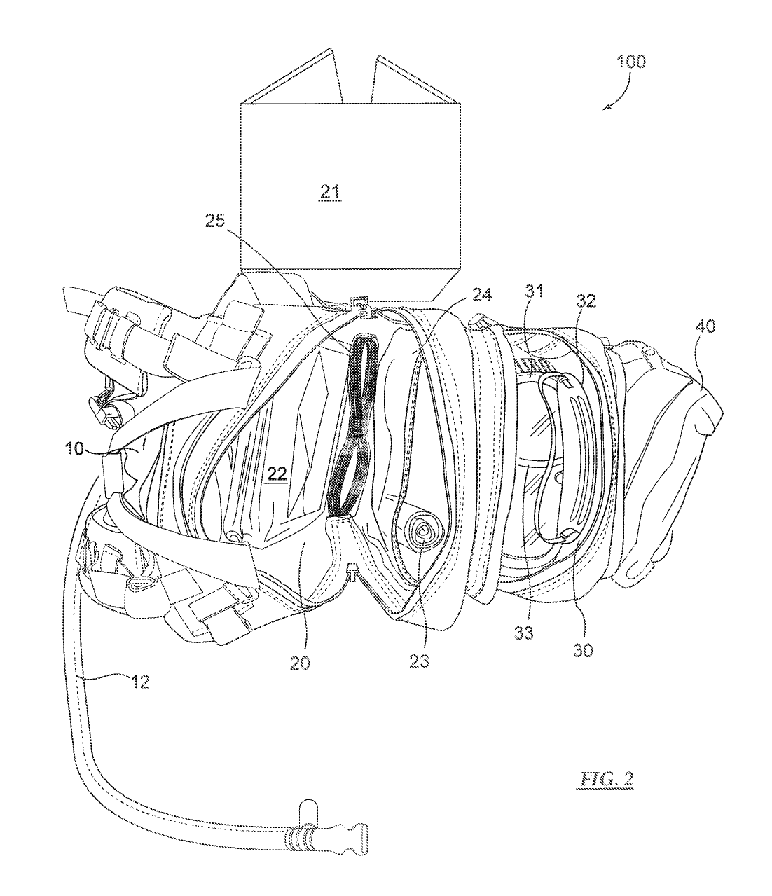

Now turning to FIG. 2, the water and ration box 21 has been removed to reveal additional contents 22, 23, 25 to the second compartment 20. Such contents initially include a lightweight, fire resistant tube tent 22 with cord (to substitute for rigid tent poles). The tube tent 22 may further be described as having the negative limitation of being devoid of tent poles. Should the disaster event result in a loss of overnight shelter, the tent would be quite useful to protect users from the weather elements. Possible scenarios for being without shelter include days following tornados or earthquakes. Also configured to the second compartment 20 are several trash bags 23 rolled tightly to conserve space. The garbage bags can be handy in an emergency situation as a makeshift tarpaulin, ground moisture barrier, or simply more storage for other items/provisions. Also configured to the second compartment 20 is an inner mesh pocket 24 to receive the rolled trash bags 23 as shown. The mesh pocket is further configured to receive nylon cord 25 to be used as a tie-down. The braided cord 25 further has multiple removable inner stands for added versatility. More specifically, the inner strands can be removed in the event that the strands are needed for multiple purposes. Various zip ties (not shown) are also included to the inner mesh pocket 24.

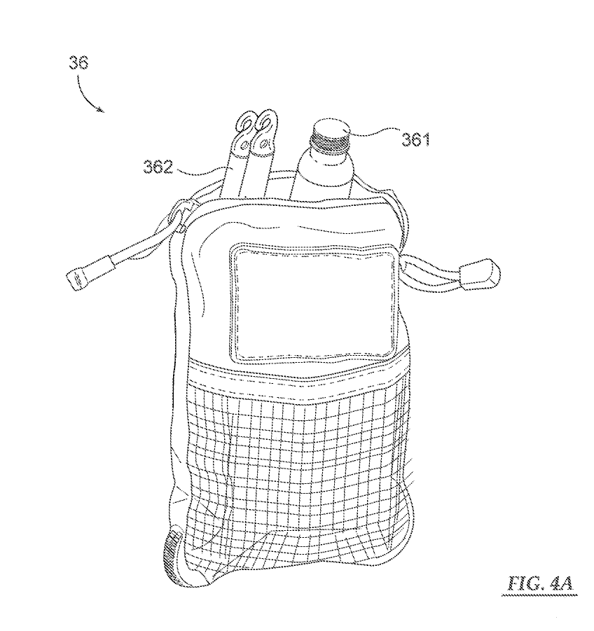

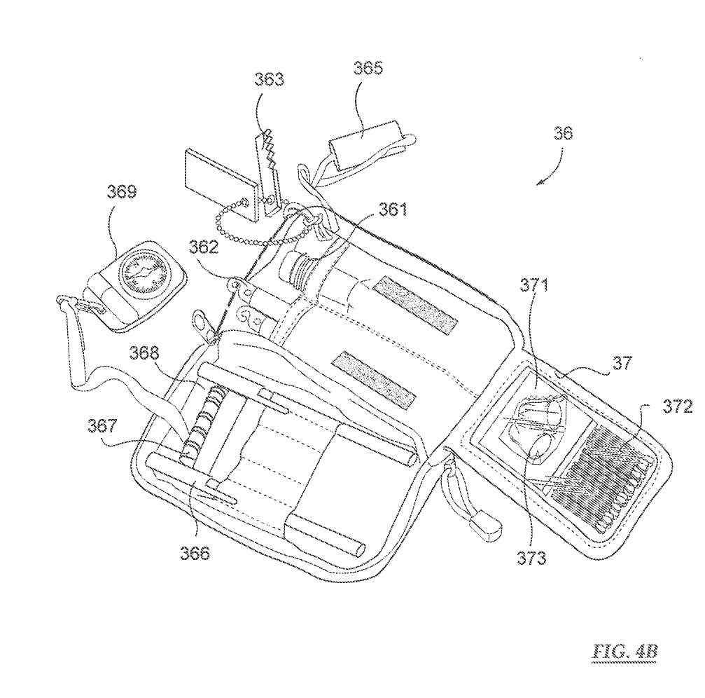

Also referring to FIG. 2, a third compartment 30 is provided flanked by second 40 and fourth 40 compartments. Initially, goggles 31 and breather masks 32 are provided to protect the wearer from airborne particulate matter. The goggles 31 and/or masks 32 may be further useful in rescue and recovery operations following, for example, a terrorist attack (urban explosion) or an earthquake. The masks 32 and goggles 31 are bound compactly with a rubber band 33 to save in space (however, the illustration shows the items side by side). Still further configured to the third compartment 30 is an outwardly opening zipper pack 36 with tool contents therein, as further illustrated in FIG. 4A and FIG. 4B.

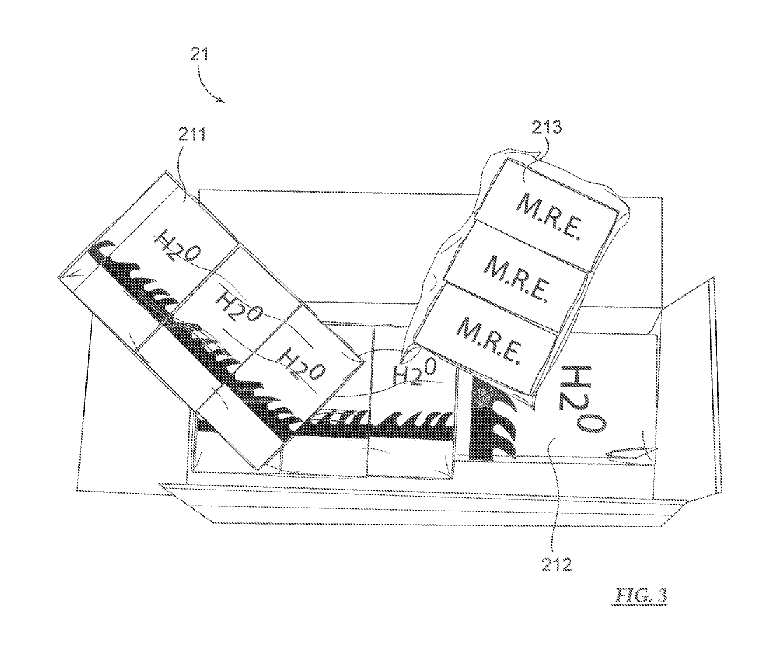

With reference to FIG. 3, a closer view of water and ration contents 21 is illustrated. More particularly, the contents are six 250 ml, 5-year shelf life, emergency drinking water cartons 211. A single, one liter package 212 of similar water is also provided to the cardboard box package. Also as shown, five-year shelf life rations 213 are provided totaling 2,400 calories of nourishment.

Regarding FIG. 4A and FIG. 4B, a relatively compact zipper pack 36 is illustrated in closed and open positions respectfully. As stated, the pack 36 is removably stowed in the third compartment 30. Further, the pack 36 has convenient storage on a right side for: emergency drinking water germicidal tablets 361, glow sticks 362, a small saw 363, and a small roll of duct tape 365. An additional outwardly opening flap 37 includes a tinder kit 373 with lighting instructions. The flap also provides waterproof matches 372. Ear plug 373 are also provided to assist sleeping in an evacuation shelter. The left half includes pen 366 and paper 367, a reflective and insulative tarp, and a compass 369. Reflectivity may be important for signaling help in a situation where natural disaster survivors (e. g. hurricane, flash flood, or tsunami) are stranded in a roof top.

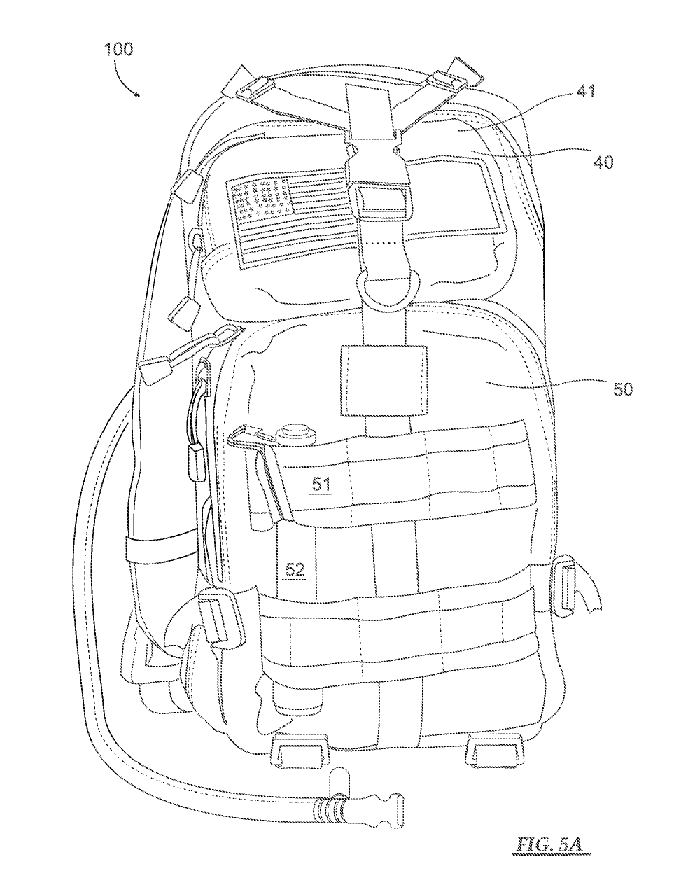

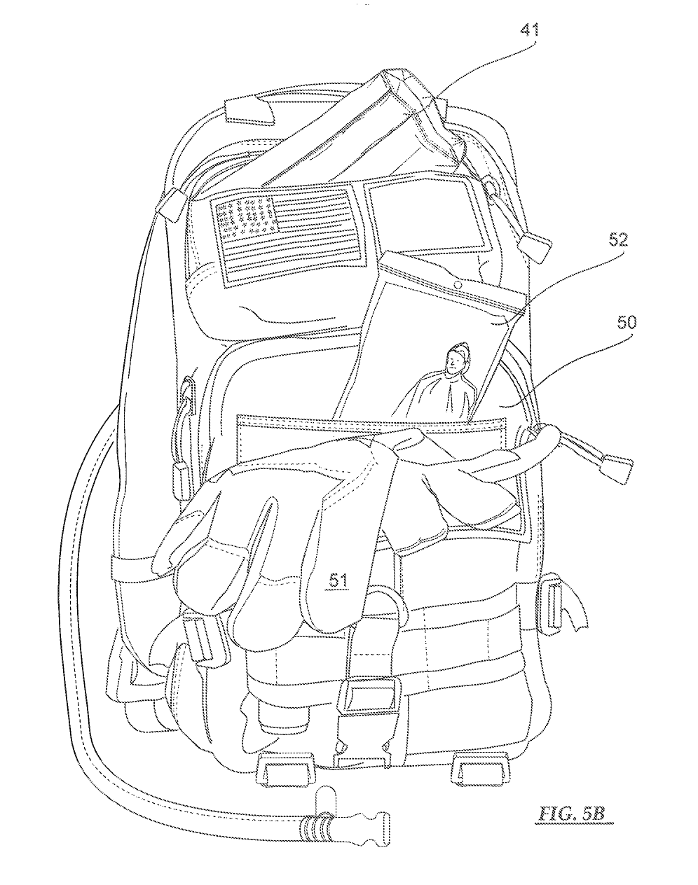

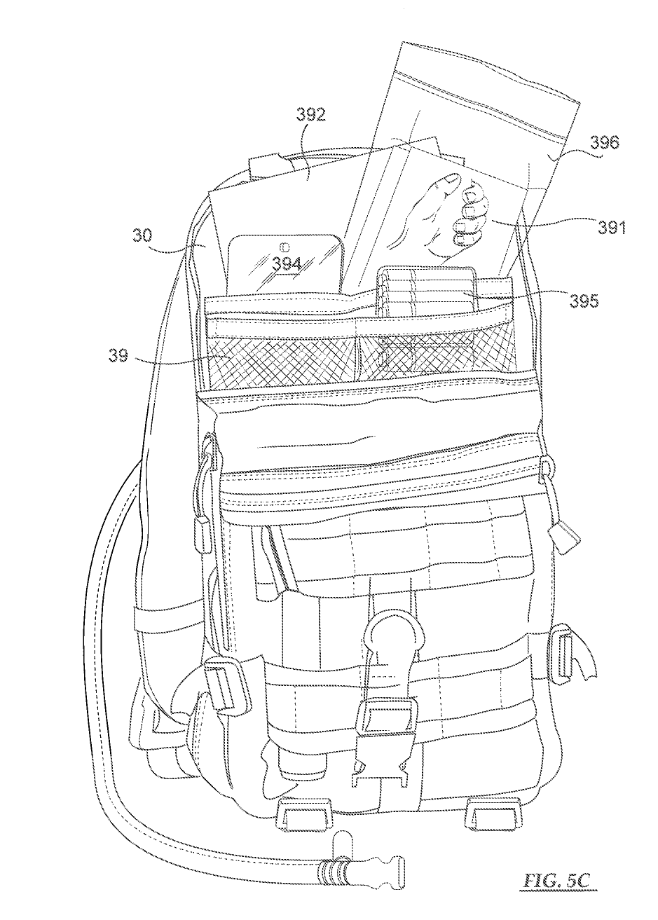

FIG. 5A is illustrative of a front of the first preferred backpack 100. A fifth compartment 50 is further provided and configured so that the fourth 40 compartment sits above the fifth 50 compartment wherein together 40, 50, they are located adjacent to the third compartment 30. As stated, a first aid kit 41 is provided to the fourth 40 compartment. More specifically, the first aid kit 41 and the fourth compartment 40 are ideally sized for a snug fit. Also according to FIG. 5A, the fifth compartment 50 has loops 51 configured to an outer portion of said compartment, the loops 51 for retaining a flashlight 52 or similarly shaped items.

Turning to FIG. 5B, the fifth compartment 50 stows work gloves 51 useful in rescue and recovery operations. For example, in an urban earthquake or a terrorist attack such as 9/11, survivors and those searching for survivors would find the gloves quite indispensable for handling sharp debris. Further to the fifth 50 compartment, an inner pocket is provided thereto having a compact, hooded poncho 52, as shown.

FIG. 5C more specifically shows the contents of a special mesh pocket 39 configured to the third compartment 30. The mesh pocket 39 is bifurcated to a front portion and a back portion. As shown, the pocket 39 contains hand warmers 391 and a survival guide book 392. The front portion has a multi-functional tool and mirror 394 on one half and batteries 395 on another half. Also provided to the third compartment 30 is an insulated pocket sized sleeping bag 396 offering protecting from rain, wind and cold temperatures.

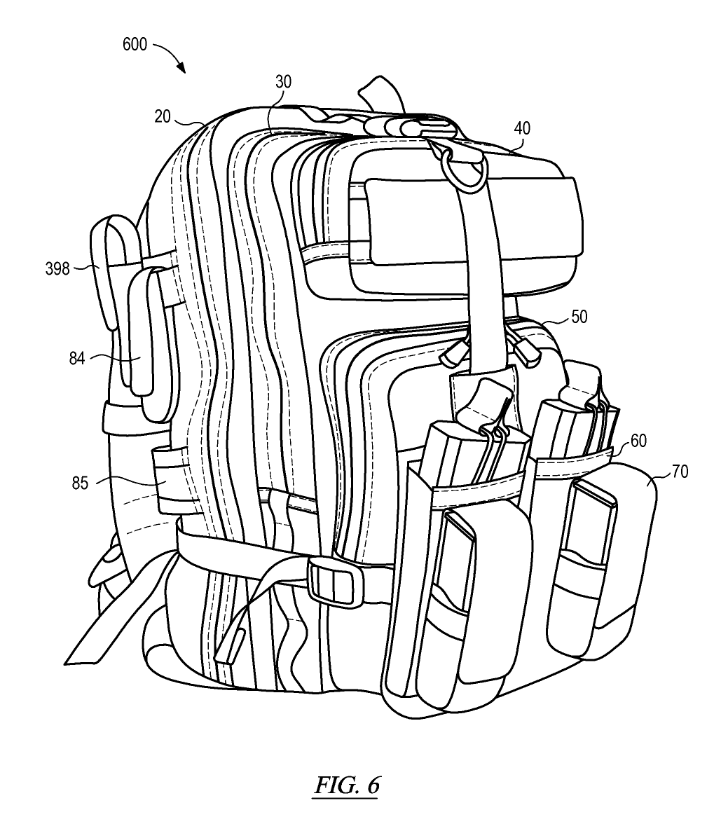

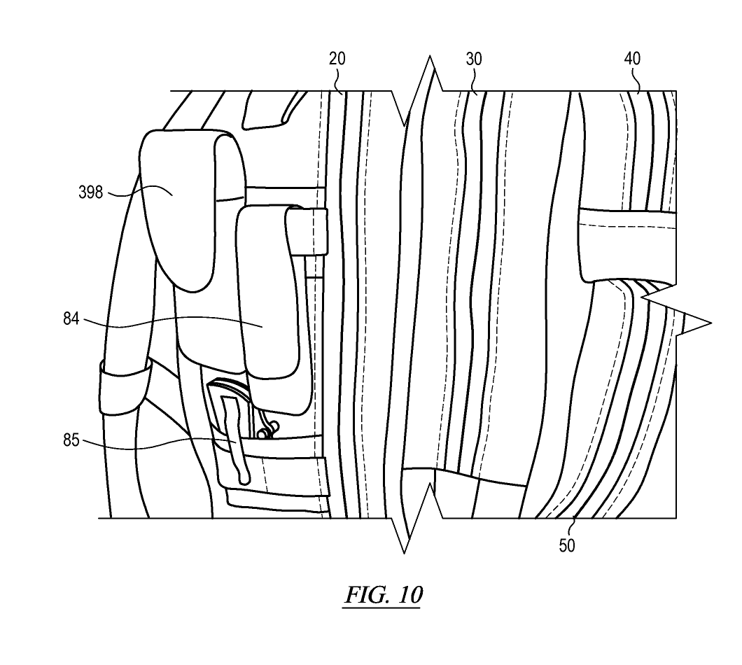

With regard to FIG. 6, a side perspective view of an active shooter response backpack 600 is provided showing four adjacent compartments (first 10 second 20 third 30 and fourth 40) with a first compartment 10 being closest to a wearer's back and other compartments 20, 30 and 40 adjacent thereto. A first compartment 10 (not shown) holds a hydration bladder 11 (not shown) having a tube 12 (not shown) for accessing water therein without having to remove the bladder 11 from the compartment 10. In a preferred embodiment, the bladder 11 holds approximately 2.5 liters of liquid. Also shown are weapon magazine pouches positioned adjacent to a fifth compartment 50. Two pistol pouches 70 and two M4 pouches 60 are shown. Located on the side of the active shooter response backpack 600 between compartment 10 and compartment 20 are a folding knife 85 and multi tool 398. Reference is made to FIG. 10 for a closer view of multi tool 398, folding knife 85 and tactical flashlight (not shown) in holster 84.

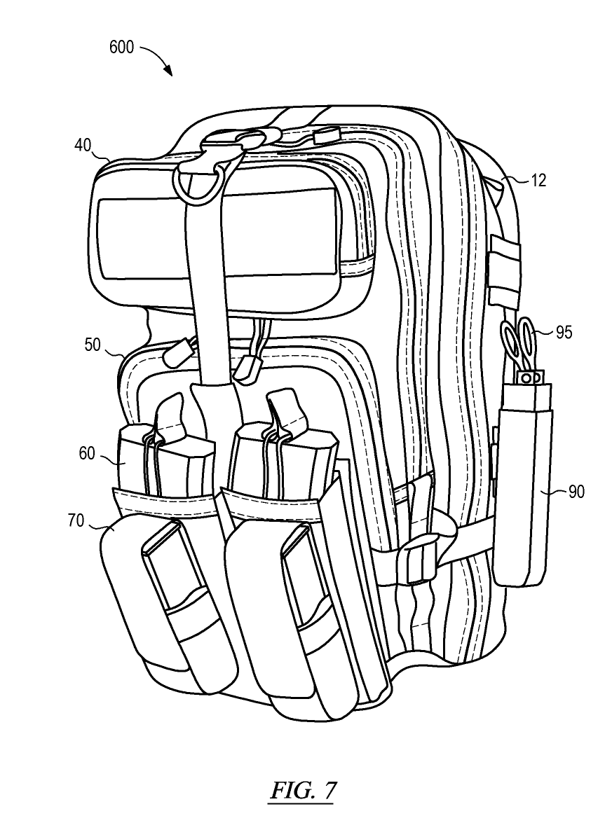

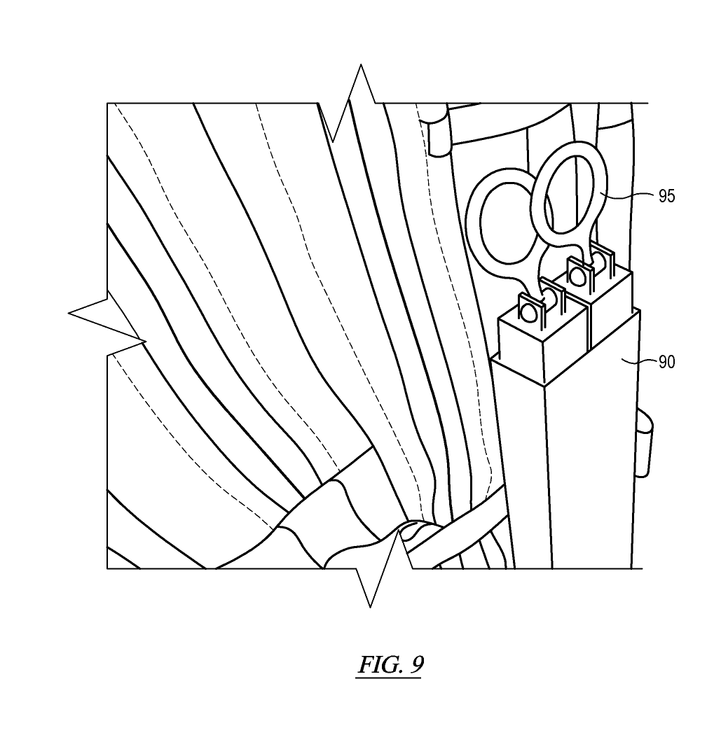

With regard to FIG. 7, an alternate side perspective view of an active shooter response backpack 600 is provided. In addition to the features illustrated in FIG. 6, disposable restraints with pull-rings 95 and restraint holster 90 are shown located on the side of the active shooter response backpack 600 between compartment 10 and compartment 20. Reference is made to FIG. 9 for a closer view of disposable restraints 95 and restraint holster 90.

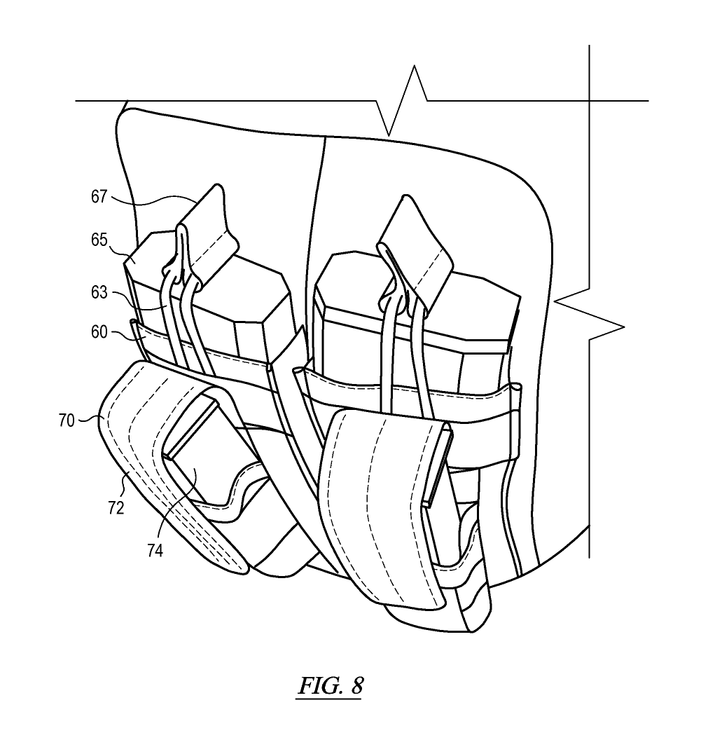

With regard to FIG. 8, shown is a close-up view of the weapons magazine pouches as illustrated in FIG. 6. The two M4 pouches 60 are located adjacent to compartment 50 and positioned side by side. In addition to each M4 pouch 60 is a double bungee cord 63 which secures each M4 magazine 65 within each M4 pouch 60. Each double bungee cord 63 further includes a canvas tab 67 for ease if accessing each double bungee cord 63 in order to release each M4 magazine 65 from pouch 60. Two pistol pouches 70 are positioned adjacent to and in front of the two M4 pouches 60, the pistol pouches 70 each having a canvas strap 72 with a magnetic closure for securing an ammunition cartridge 72.

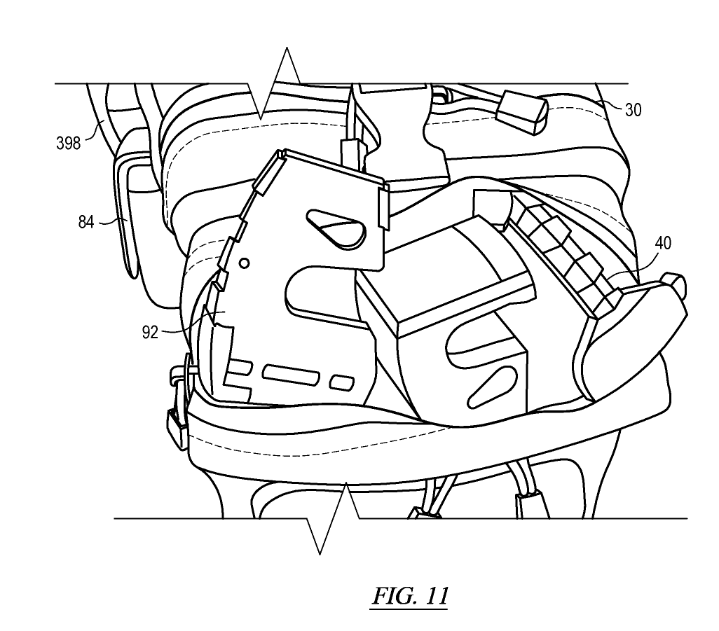

With regard to FIG. 11, shown is compartment 40 having a plurality of universal door stops 92. As illustrated, at least three universal door stops 92 fit readily into compartment 40 for easy access.

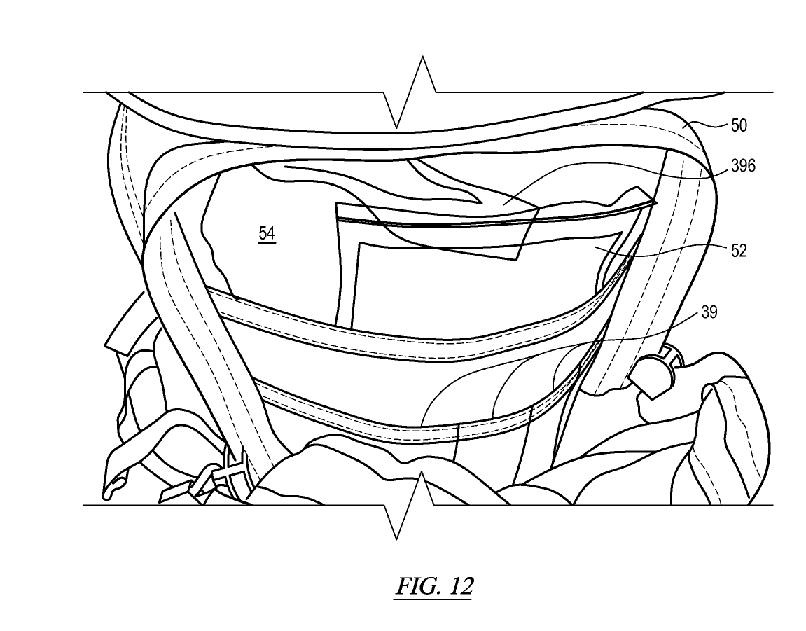

With regard to FIG. 12, shown is compartment 50 having a compact, hooded poncho 52, dust/particulate/contagion masks 54, and an insulated pocket sized sleeping bag 396. In addition, compartment 50 further includes a plurality of pockets 39 for placement of various smaller sized items.

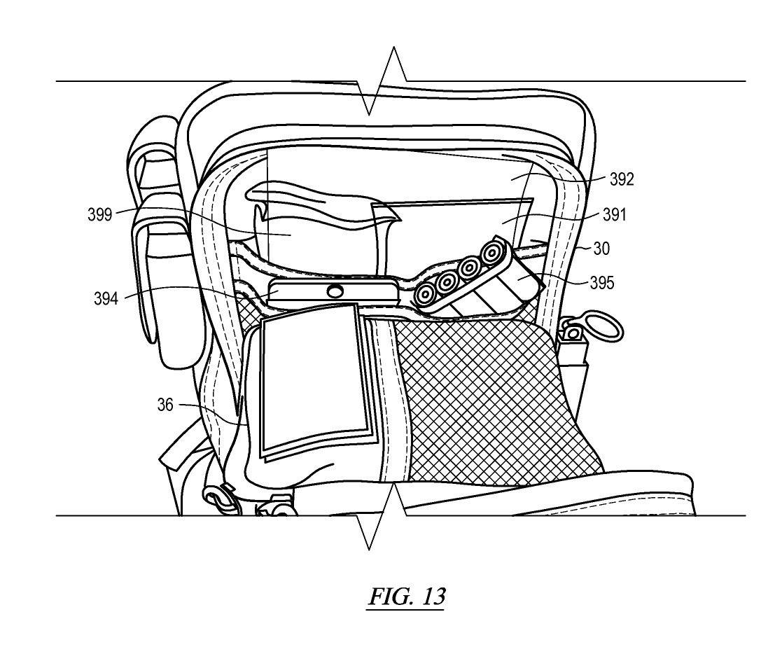

With regard to FIG. 13, shown is compartment 30 having a survival guide 392, multi tool user's manual 399, and chemical hand warmers 391 fitting in the rear most pocket of compartment 30. In additional pockets adjacent to the rear most pocket of compartment 30 are lithium batteries 395 and an acrylic mirror 394. Compartment 30 further includes a fully contained compact survival kit 36 which fits snuggly in from of the additional pockets of compartment 30. Although not shown, the fully contained compact survival kit 36 includes, but is not limited to, an emergency blanket, paper, pens, chemical lights, disposable butane lighter, magnesium fire starter, water proof matches, emergency fire starter, 50″ duct tape, compass, whistle and water purification tablets.

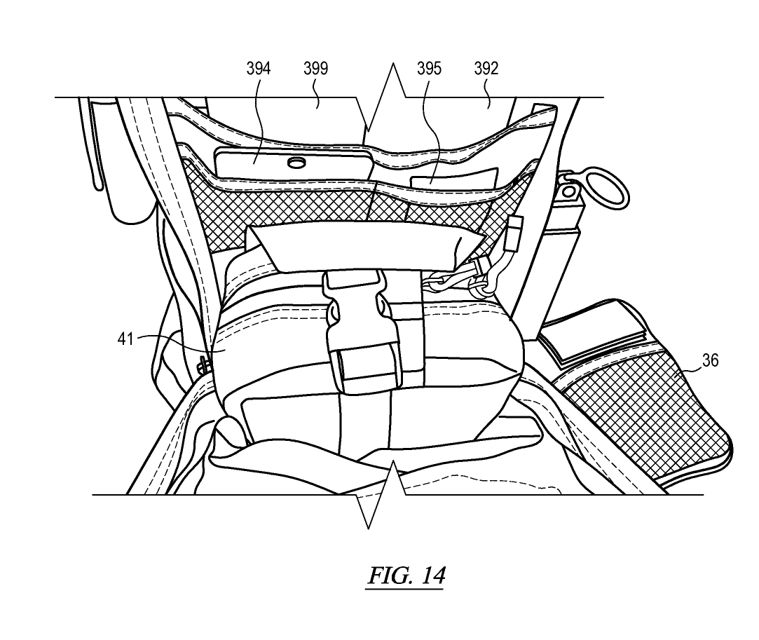

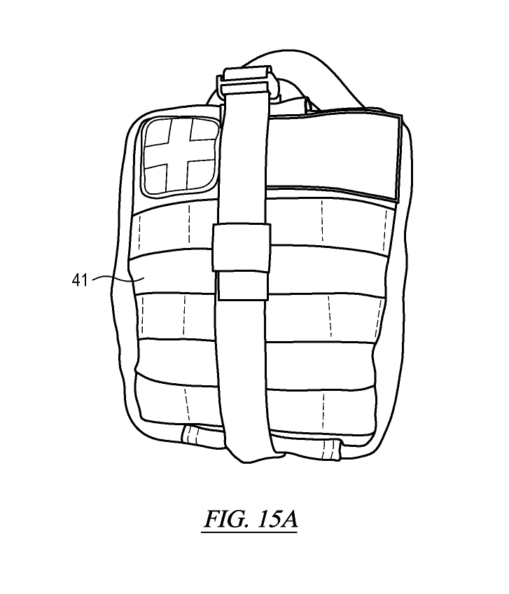

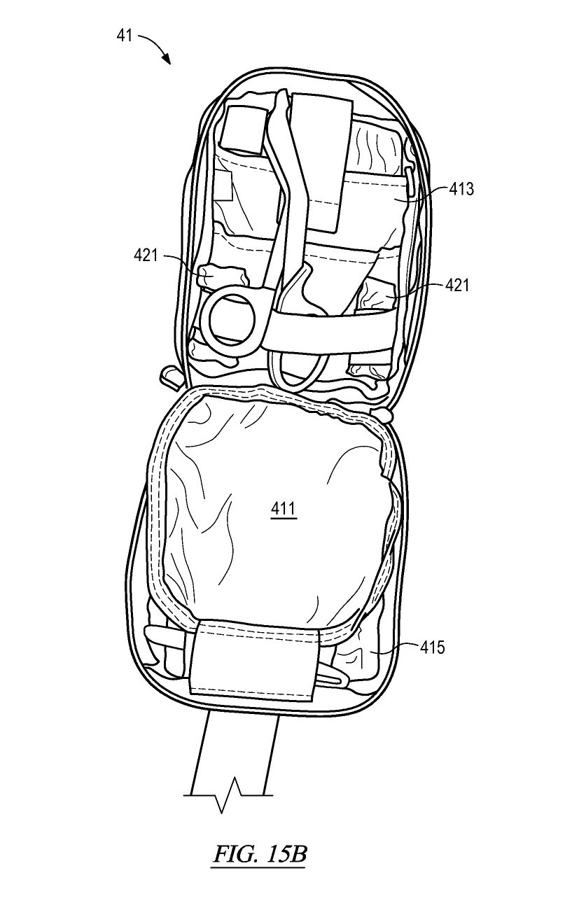

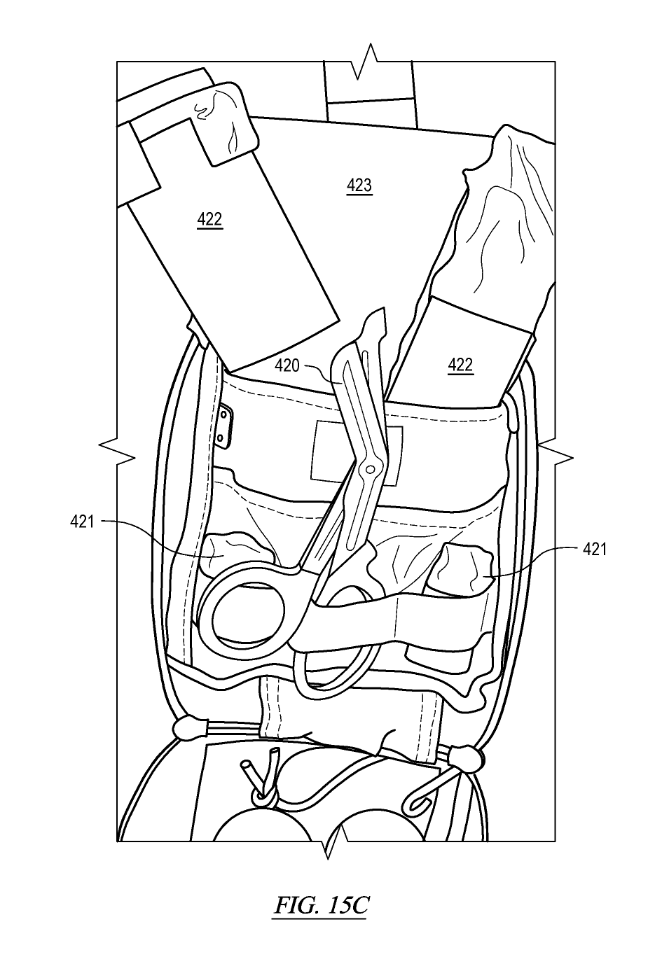

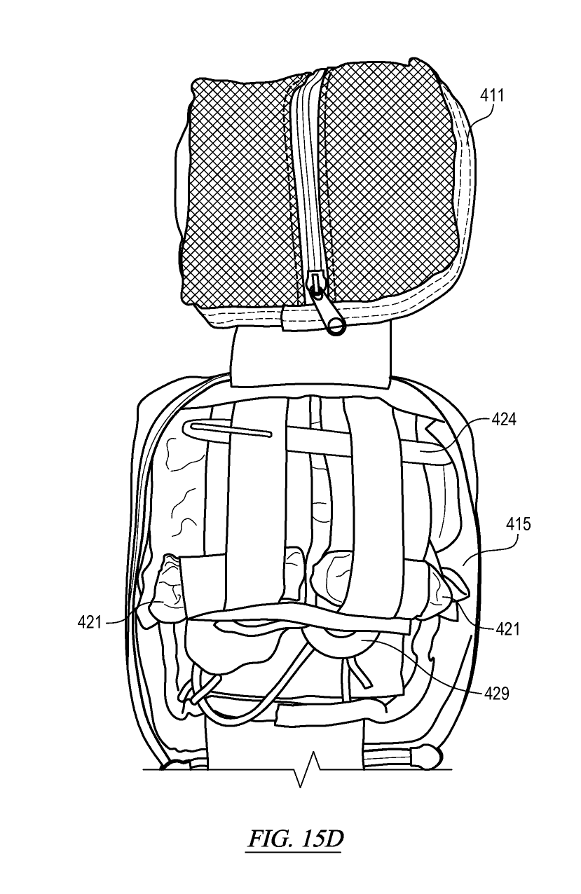

With regard to FIG. 14, shown is compartment 30 further including a trauma kit 41 which fits snuggly underneath the fully contained compact survival kit 36. FIG. 15A shows the relative size of trauma kit 41 once removed from compartment 30.

With regard to FIG. 15B, shown is the interior of trauma kit 41 having a first compartment 413 for securing trauma supplies such as shears 420 and nitrile gloves 421. Also shown is a central pocket 411 which folds out to allow access to compartment 415. Compartment 413 further includes tourniquets 422 and hemostatic gauze 423 as illustrated in FIG. 15C. With regard to FIG. 15D, shown is a second compartment 415 for securing additional trauma supplies such as nitrile gloves 421, permanent marker 424 and surgical tape 425. Although not shown, trauma kit 41 further includes 6″ combat dressing, compressed gauze and a suture kit.

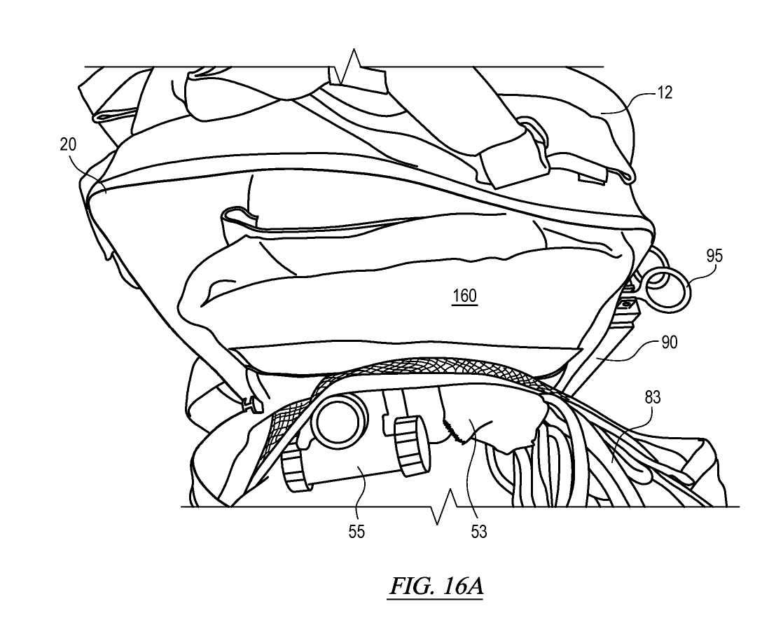

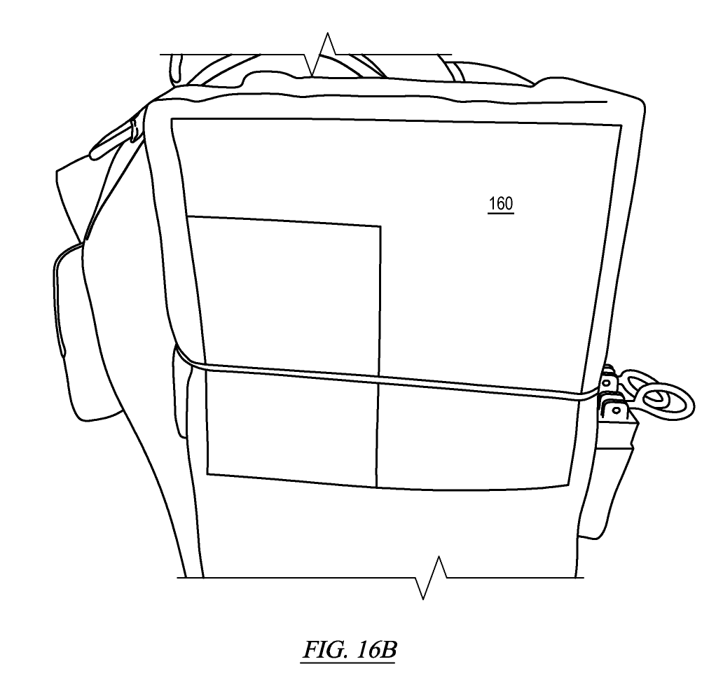

With regard to FIG. 16A, shown is compartment 20 having a pole-less evacuation litter 160 located in the position closest to the wearers back in compartment 20. Compartment 20 further includes a mesh pocket for holding a headlamp 55, refuse bags 53 and a 50′ paracord. FIG. 16B shows the relative size of pole-less litter evacuation litter 160 as it fits in compartment 20.

One embodiment of the present invention provides an active shooter response system including tools and gear necessary for law enforcement to approach an active shooter scenario while also providing crucial retrievability of time critical items.

Another embodiment of the present invention provides an active shooter response system wherein the response system provides first responders with the equipment to engage an assailant, manage a scene, facilitate civilian evacuation and render emergency first aid care on site. In a preferred embodiment, first aid items and protective gear are readily available at the top of the fourth compartment to ensure easy access in the case of urgent need. In another preferred embodiment, camping and ration items are located deeper in their respective compartments.

Yet another embodiment of the present invention provides a survival kit comprising a specially designed and arranged backpack, the backpack comprising: a plurality of compartments with a first compartment being closest to a wearer's back, the first compartment including a hydration bladder having a tube for accessing water therein without having to remove the bladder from the first compartment; a second compartment including a pole-less evacuation litter, headlamp and paracord; and a third compartment including a trauma kit and a fully contained compact survival kit.

A further embodiment of the present invention provides a fourth compartment adjacent to the third compartment, the fourth compartment containing a plurality of universal door stops. A fifth compartment is located underneath the fourth compartment, wherein the fifth compartment includes at least dust/particulate/contagion masks and a hooded poncho.

A further still embodiment of the present invention provides ammunition magazine pouches adjacent to the fifth compartment, wherein the magazine pouches accommodate at least M4 magazines and at least two pistol magazines.

A preferred embodiment of the present invention provides a survival kit comprising a specially designed and arranged backpack, wherein items in the backpack are arranged according to the typical order of time criticality of use in order to minimize time for first responders to locate critical items when first arriving on the scene of an emergency situation.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

While the particular Compositions and Methods for Arrangement of Survival Materials as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

What is claimed is:

1. An active shooter response system comprising a specially designed and arranged backpack, the backpack comprising:
a plurality of compartments with a first compartment being closest to a wearer's back, the first compartment including a hydration bladder having a tube for accessing water therein without having to remove the bladder from the first compartment;
a second compartment including at least a pole-less evacuation litter, headlamp and paracord; and
a third compartment including a trauma kit and a fully contained compact survival kit.

2. The active shooter response system comprising a specially designed and arranged backpack of claim 1, the backpack further comprising:
a fourth compartment adjacent to the third compartment, the fourth compartment containing plurality of universal door stops; and
a fifth compartment located underneath the fourth compartment, the fifth compartment including at least dust/particulate/contagion masks and a hooded poncho.

3. The active shooter response system comprising a specially designed and arranged backpack of claim 2, the fifth compartment further comprising:
an inner pocket including a cocoon sleeping bag; and
loops configured to an outer portion of the fifth compartment, the loops securing a plurality of ammunition magazine pouches to the front of the backpack for easy access.

4. The active shooter response system comprising a specially designed and arranged backpack of claim 1, the third compartment further comprising:
a survival guide;
a multi tool user's manual;
chemical hand warmers;
lithium batteries; and
an acrylic mirror.

5. The active shooter response system comprising a specially designed and arranged backpack of claim 1, the compact survival kit further comprising:
an emergency blanket;
paper and pens;
chemical lights;
a disposable butane lighter;
a magnesium fire starter;
water proof matches;
an emergency fire starter;
50″ duct tape;
compass and whistle; and
a plurality of water purification tablets.

6. The active shooter response system comprising a specially designed and arranged backpack of claim 1, the trauma kit further comprising:
shears;
a plurality of nitrile gloves;
tourniquets;
hemostatic gauze;
a permanent marker;
surgical tape;
6″ combat dressing and compressed gauze; and
a suture kit.

7. The active shooter response system comprising a specially designed and arranged backpack of claim 1, the outer surface of the backpack further comprising:
a restraint holster with tri-fold disposable restraints with pull rings;
a multi tool;
a handheld tactical flashlight; and
a folding knife with integral glass breaker and seatbelt cutter.
